(19) United States
(12) Patent Application Publication
(10) Pub. No.: US 2022/0403032 A1
(43) Pub. Date: Dec. 22, 2022

(54) THERAPY FOR DIABETES USING STEM CELL MIGRATION AGENT

(71) Applicant: Biozipcode, Inc., Otsu-shi, Shiga (JP)

(72) Inventors: Hideto Kojima, Otsu-shi, Shiga (JP); Tomoya Terashima, Otsu-shi, Shiga (JP); Miwako Katagi, Otsu-shi, Shiga (JP)

(21) Appl. No.: 17/769,710

(22) PCT Filed: Oct. 16, 2020
(86) PCT No.: PCT/JP2020/039045

§ 371 Date: Apr. 15, 2022

(30) Foreign Application Priority Data
Oct. 18, 2019 (JP) 2019-191369

(51) Int. Cl. C07K 16/28; A61K 38/19; A61K 31/395; A61K 31/5377; A61K 31/517; A61K 31/506; A61P 3/10; G01N 33/569; G01N 33/68

(52) U.S. Cl. CPC C07K 16/2836; A61K 38/193; A61K 31/395; A61K 31/5377; A61K 31/517; A61K 31/506; A61P 3/10; G01N 33/56966; G01N 33/6872

(57) ABSTRACT

The present disclosure provides a therapy for diabetes that targets abnormal stem cells in combination with stem cell migration. In one embodiment, the present disclosure provides a therapy for diabetes and/or diabetes-related diseases and disorders and/or symptoms that targets abnormal stem cells in combination with stem cell migration. In one embodiment, the present disclosure provides diagnosis of diabetes and/or diabetes-related diseases and disorders and/or symptoms, or the risk thereof, using abnormal stem cell migration and/or residence as an indicator.

antibody, while normally functioning stem cells may regenerate islets.

### EXAMPLE 9: COMBINATION TREATMENT OF A SUPPRESSING AGENT AND A MIGRATION AGENT FOR STEM CELLS IN NOD MICE

With spontaneous type 1 diabetes model mice (NOD mice), the therapeutic effect of a combination of a suppressing agent and a migration agent for abnormal stem cells was tested.

Method

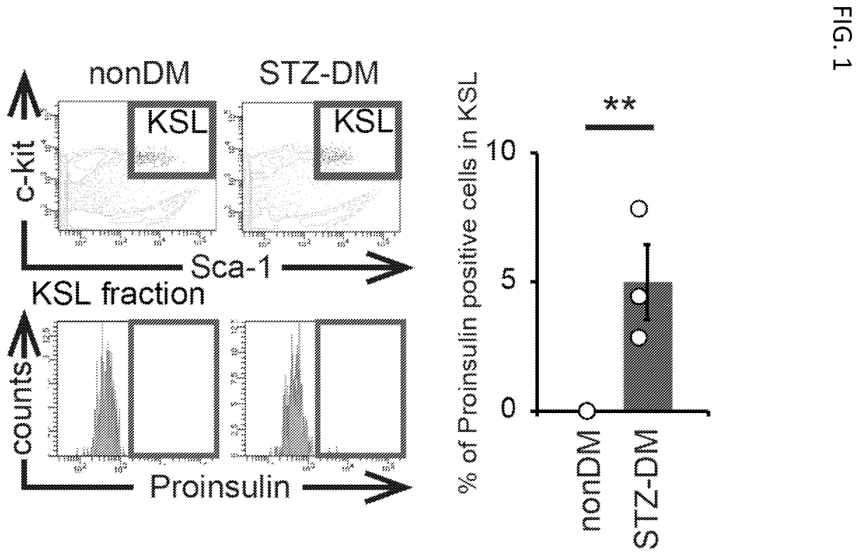

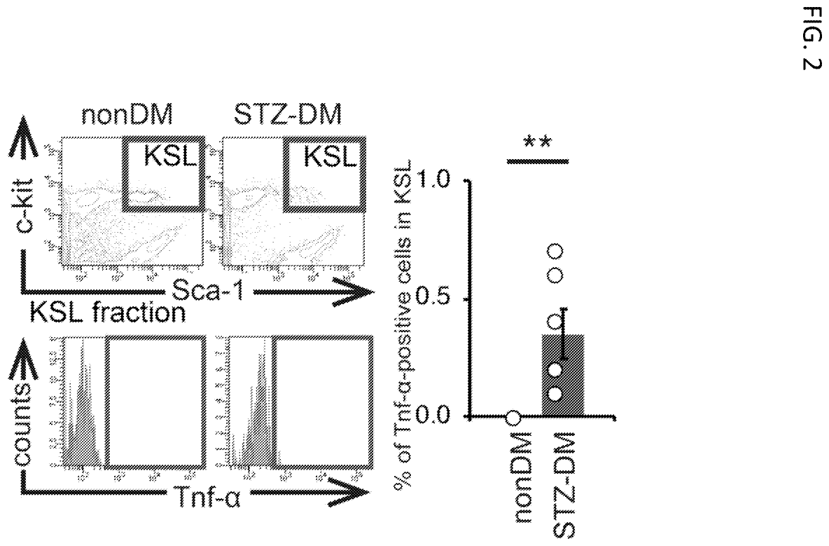

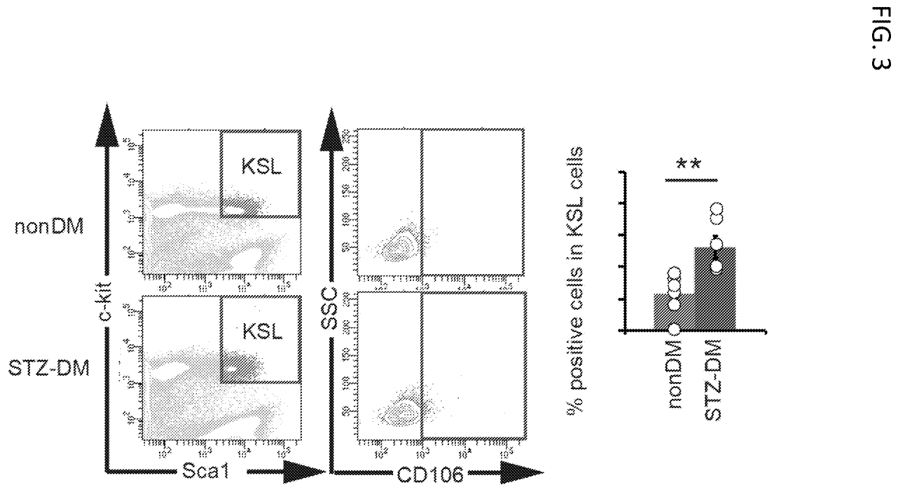

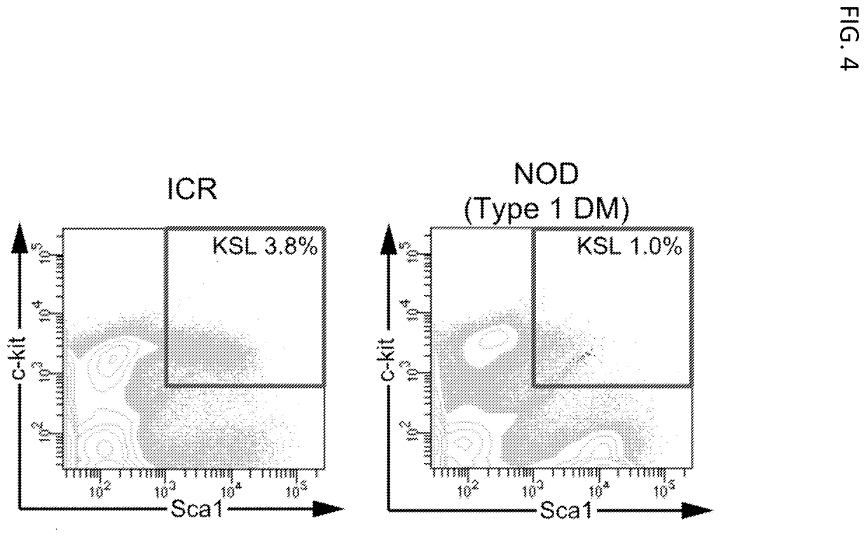

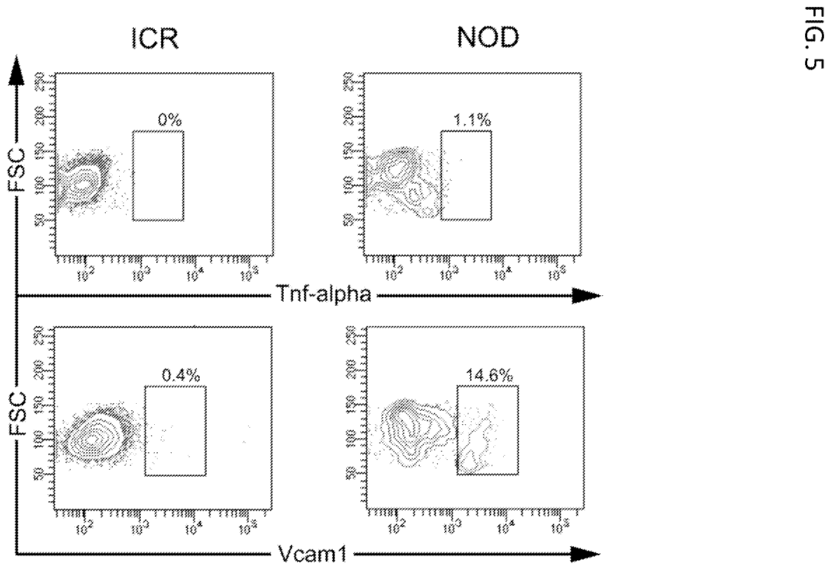

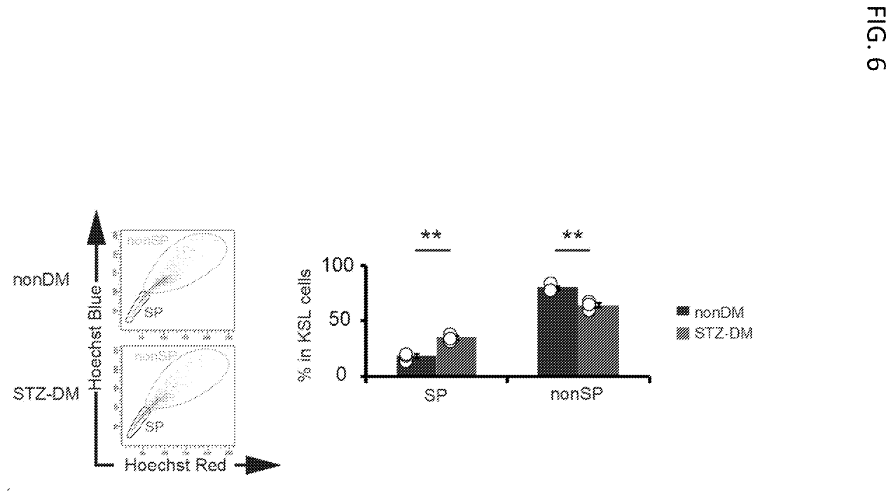

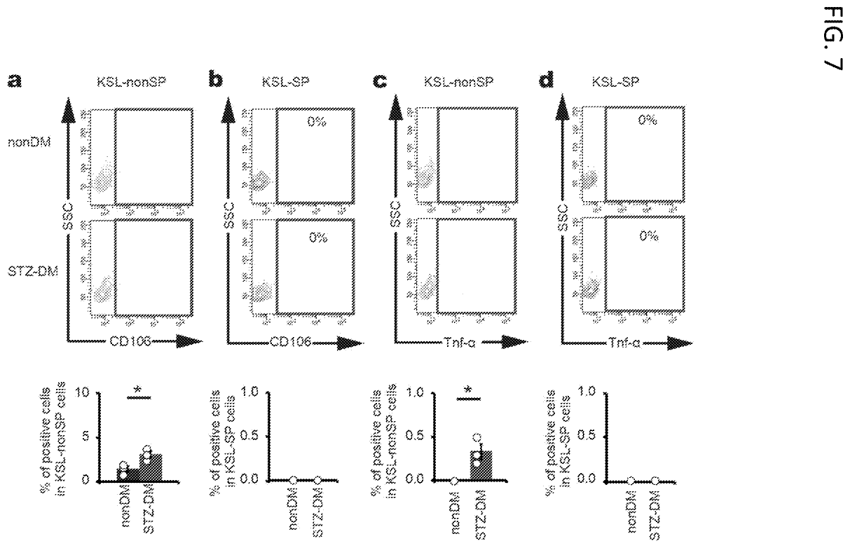

NOD mice, which develop spontaneously type 1 diabetes models, were purchased from CLEA Japan, Inc. (Osaka), and the blood glucose level and body weight of the mice were measured every two weeks. For the mice in which their blood glucose levels increased, was observed, AMD3100 (5 mg/kg: abcam)+GROβ (0.1 mg/kg: Peprotech) was prepared with saline and then subcutaneously injected into them, and fifteen minutes later, the mice were administered anti-CD106 antibody (250 μg/mouse) via tail vein (administration interval was weekly). After the start of the therapy, the blood glucose level and body weight were measured daily (FIG. 18).

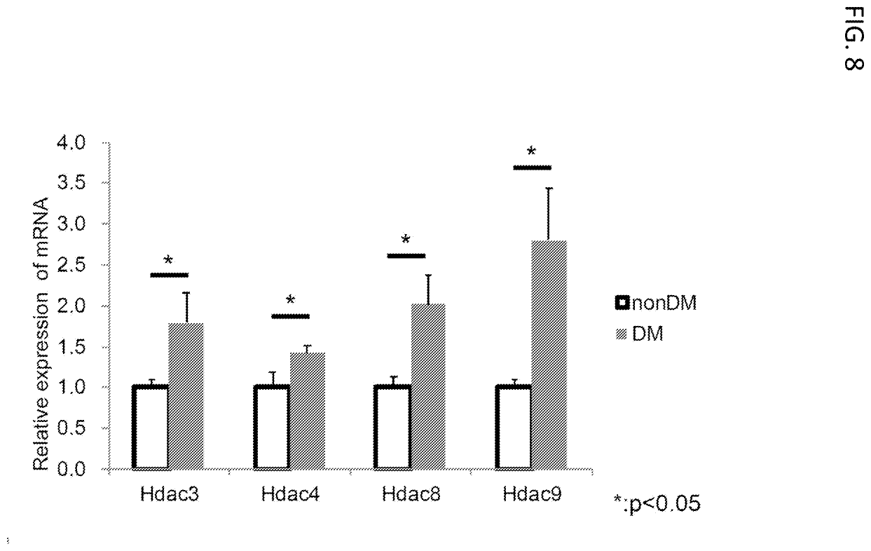

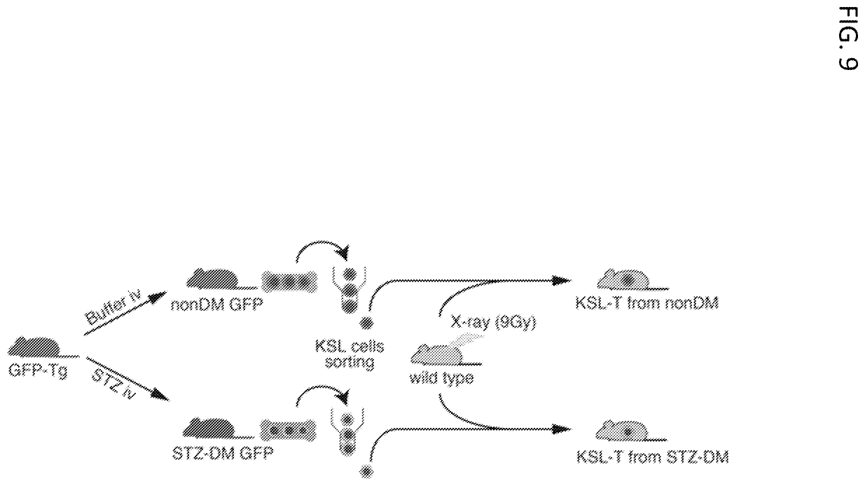

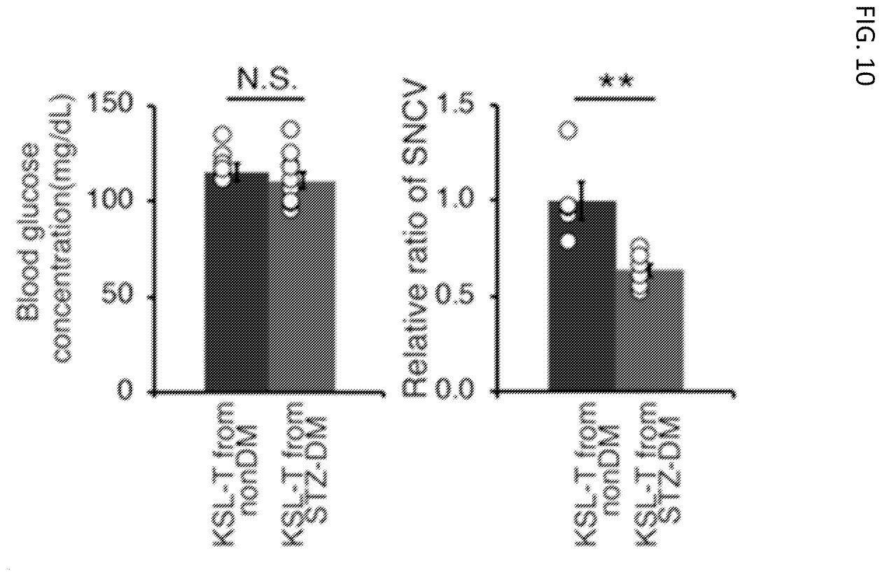

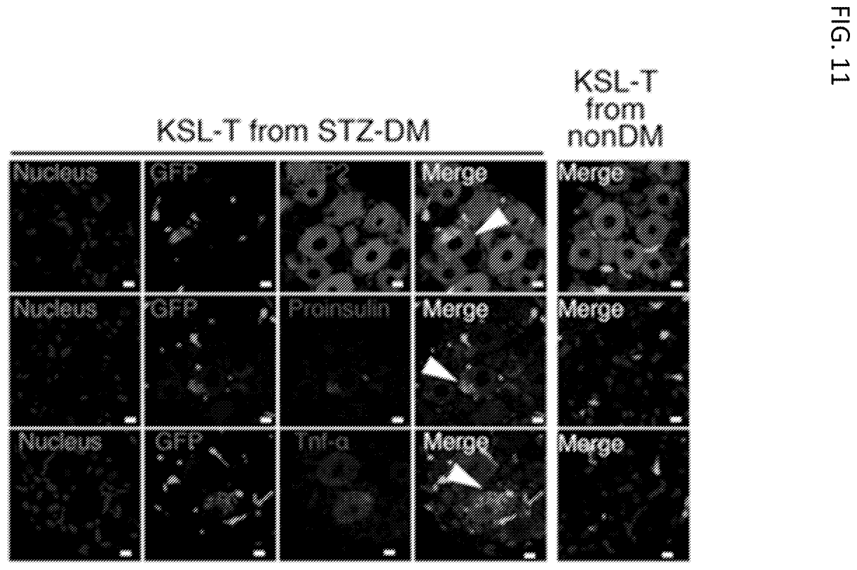

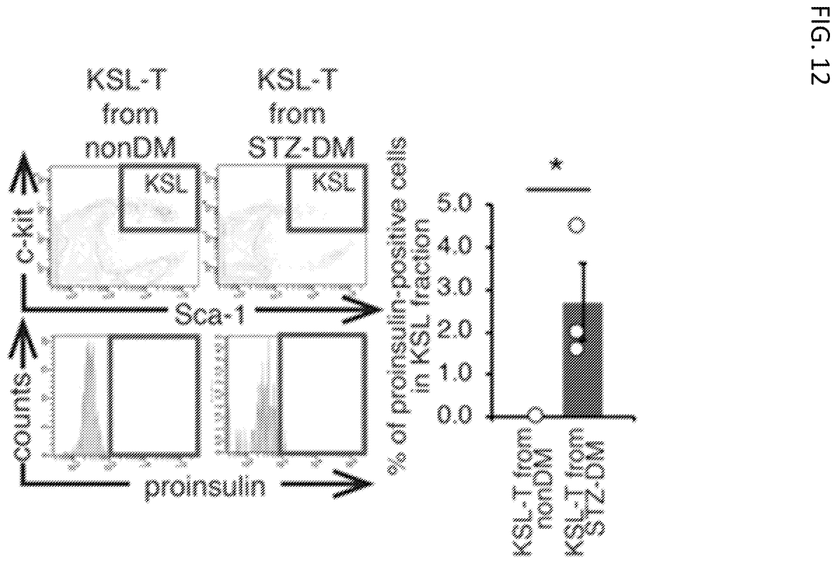

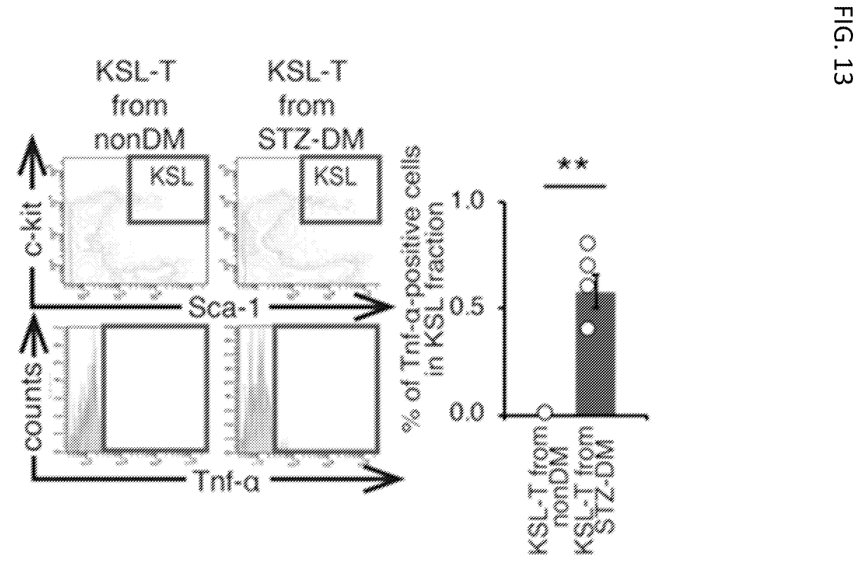

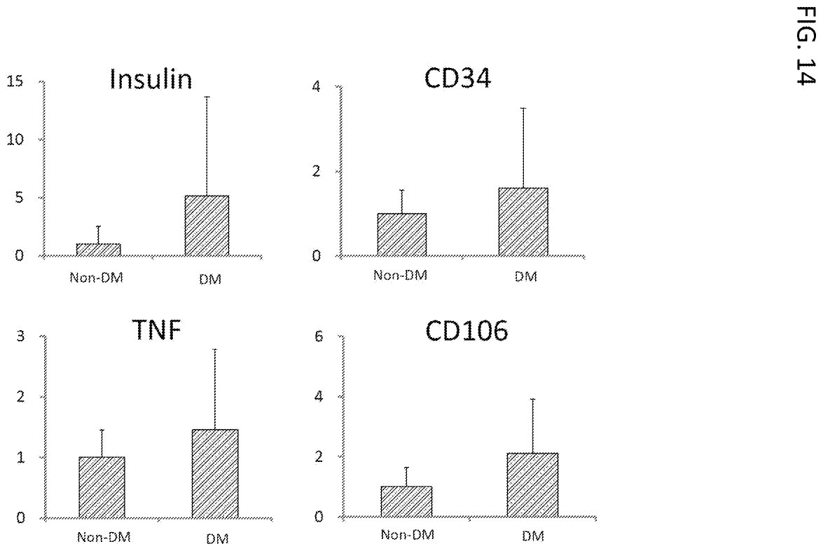

In addition, prior to the start of the therapy, in order to confirm the islet status of ICR mice, NOD mice that did not develop diabetes mellitus yet, and NOD mice that developed diabetes mellitus (all mice used were of the same age in week), perfusion fixation was performed, and immunostaining was performed as follows (FIG. 19).

Wash three times with PBS (−) for ten minutes.

Soak in a 0.3% H₂O₂ PBS (−) solution at room temperature for thirty minutes to inactivate the endogenous peroxidase.

Wash three times with PBS (−) for five minutes.

Incubate for one hour at room temperature with blocking buffer (5% normal goat serum in PBS 0.3% triton X-100).

Add a primary antibody (anti-insulin antibody: CST) and incubate at 4° C. overnight.

Wash three times with PBS (−) for five minutes.

Add ImmPRESS Reagent (Anti-rabbit: VECTOR Laboratories) and incubate at room temperature for thirty minutes.

Wash three times with PBS (−) for five minutes.

Add ImMPACT DAB substrate (VECTOR Laboratories) and allow it to react at room temperature for thirty seconds.

Add dH₂O to stop the DAB reaction.

Counter-stain with hematoxylin for thirty seconds.

Wash with tap water.

Dehydrate and infiltrate.

Images of each of the prepared slides were acquired.

Result

In the NOD mice, the islet inflammatory reaction was already very strong before the onset of diabetes mellitus and insulin staining was very low (FIG. 19). In addition, it was confirmed that the blood glucose level of the diabetes mellitus mice decreased by the above treatment (FIG. 18).

The combination therapy of the migration agent and the antibody showed a clear hypoglycemic effect. This effect is considered to be due to the restoration of pancreatic islet function by the antibody.

It has been suggested that removal of abnormal hematopoietic stem cells is also a useful therapeutic strategy for type 1 diabetes mellitus.

### EXAMPLE 10: ABNORMAL CELL IN THE BONE MARROW OF A HUMAN DIABETIC PATIENT

It was confirmed that the bone marrow of a human diabetic patient also has an abnormal cell.

The inventors studied the presence or absence of the appearance of a proinsulin positive cell in the bone marrow of patients with type 2 diabetes mellitus (DM) who were hospitalized in Shiga University of Medical Science and autopsied between Jan. 1, 2000 and Dec. 31, 2010. The tissue was embedded in paraffin in Shiga University of Medical Science, Anatomy Center. A 5 μm thick section of the paraffin-embedded sample was treated for immunohistochemistry by using avidin-biotin-peroxidase complex (ABC) method and diaminobenzidine (DAB)-nickel reaction. After the section was deparaffinized in xylene and alcohol, the section was treated with a microwave (for 10 minutes at 0.5 kW at a pH of 6.0 in 10 mmol/L of citrate buffer), followed by incubation overnight with an antibody against proinsulin (mouse monoclonal, Abcam, UK) diluted at 1:1,000 in 0.1% PBS comprising 0.3% Triton X-100 (PBST). Subsequently, treatment for immunohistochemistry was performed at 4° C. After the DAB-nickel reaction, the segment was counterstained with a nuclear fast red solution.

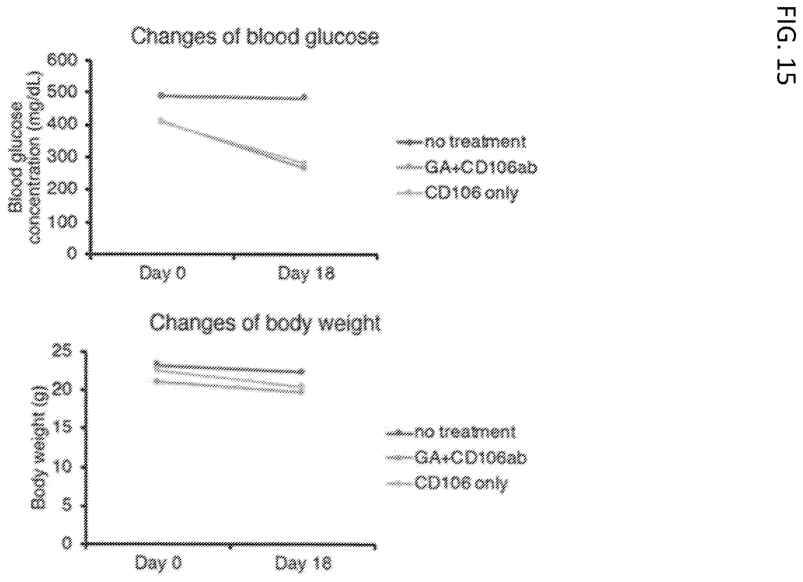

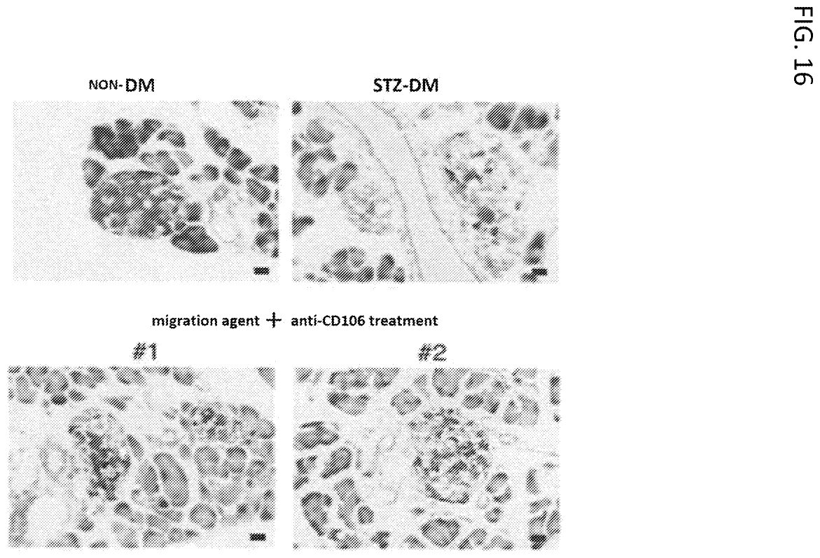

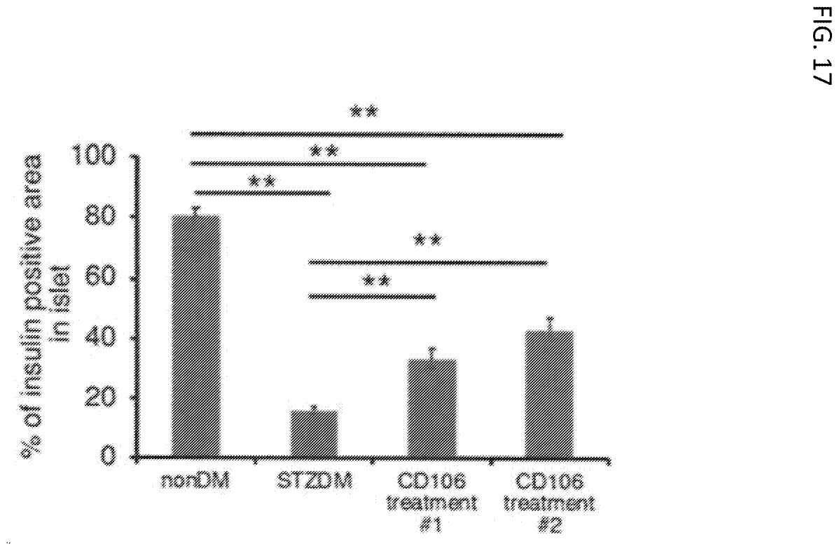

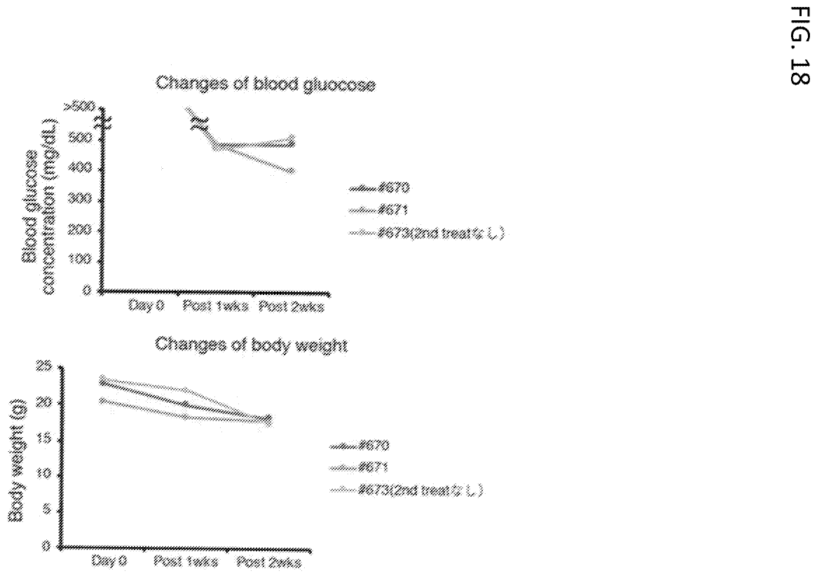

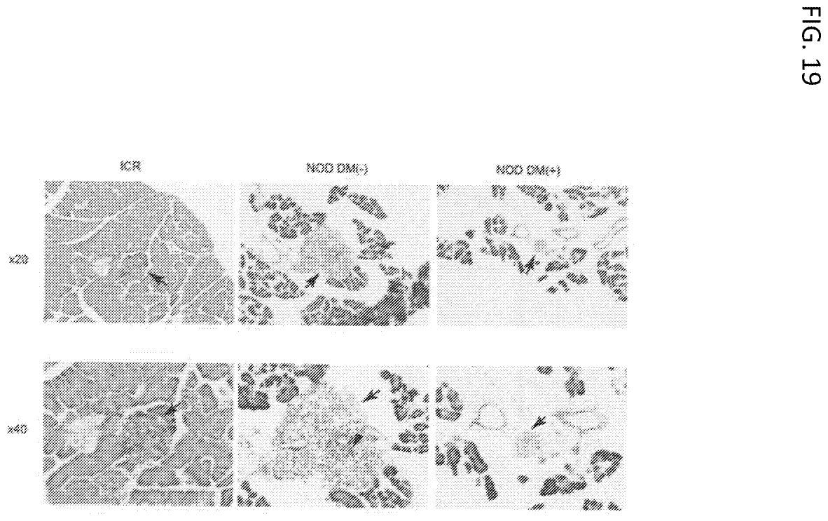

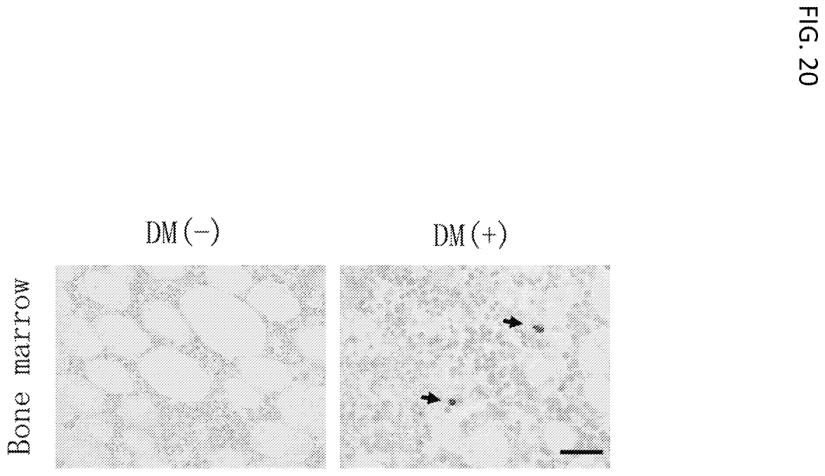

FIG. 20 shows the result. While proinsulin expression was not observed in bone marrow cells derived from patients without DM, proinsulin expression was observed in bone marrow cells derived from patients with DM. It is considered that the finding on an abnormal stem cell observed in mice is also applicable to humans.

### EXAMPLE 11: APPLICATION TO HUMANS

A bone marrow-derived abnormal hematopoietic stem cell is identified in a human diabetic patient, and diabetes mellitus is treated while targeting the abnormal hematopoietic stem cell.

Research Plan 1: Identification of a Bone Marrow-Derived Abnormal Hematopoietic Stem Cell in a Diabetic Patient

(Subject)

For a non-diabetic group, volunteers who have no prior history of impaired glucose tolerance and are not currently receiving therapy for diabetes mellitus are recruited from the staff of Shiga University of Medical Science and Shiga University of Medical Science Hospital. The blood glucose level and HbA1c are continuously measured, and those who satisfy casual blood glucose level <140 mg/dl and HbA1c<6.0% are defined as non-diabetes melitus. 20 people are registered as a control group. Those who have HbA1c that is 6.5% or greater or who are under therapy for diabetes mellitus are defined as a diabetic group. A list of patients whose sex and age are matched with the non-diabetic group is prepared based on the electronic medical record from the patients regularly attending the outpatient clinic of diabetes mellitus and endocrine internal medicine of Shiga University of Medical Science Hospital. 80 patients (40 patients with type 1 diabetes mellitus and 40 patients with type 2 diabetes mellitus) are registered at random.

(Research Method)

Medical questions are asked to the subjects, the height and body weight of the subjects are measured, a blood test and a urine test are performed, and the presence or absence of diabetes mellitus is determined. Mononuclear cells are extracted from the collected blood, CD34-labelled bone marrow progenitor cells are collected and fixed, and the form and expressed protein are identified by immunostaining. Further, after mRNA is extracted, cDNA is prepared and the amount of expression of mRNA is quantified by quantitative PCR. Specifically, the amount of expression of TNF-α mRNA and insulin mRNA or the like is measured in CD34 positive and CD106 positive (CD34/CD106) bone marrow progenitor cells in peripheral blood. The presence or absence of expression of protein in these cells is also measured. Association between the presence or absence of a diabetic complication in the diabetic patients and blood glucose control is also analyzed. A nerve conduction velocity test, an electrocardiogram R-R interval test, an ophthalmoscopy, a urinary albumin excretion rate, quantification of the amount of intraperitoneal fat using abdominal CT, a blood lipid test, an electrocardiogram, a carotid artery echo test, and a lower limb artery echo test are performed to check the presence or absence of diabetic neuropathy, retinopathy, nephropathy, fatty liver, and dyslipidemia, which are representative complications, and macrovasculopathy.

(Prediction of Results)

(1) While expression of TNF-α mRNA and insulin mRNA is observed in CD34/CD106 bone marrow progenitor cells in peripheral blood of the non-diabetic group and the type 2 diabetic group, the amount of expression increases in the type 2 diabetic group. On the other hand, in the type 1 diabetic group, while expression of TNF-α mRNA increases in the CD34/CD106 bone marrow progenitor cells as compared to non-diabetes melitus, insulin mRNA is not expressed at all.

(2) There are very few cells expressing TNF-α protein and proinsulin protein in CD34/CD106 bone marrow progenitor cells in peripheral blood of the non-diabetic group. Meanwhile, cells expressing both of the proteins increase in type 2 diabetes mellitus. On the other hand, in type 1 diabetes mellitus, while cells expressing TNF-α protein increase, there is no cell expressing proinsulin.

(3) In the type 1 diabetic patients, onset of diabetic neuropathy, retinopathy, nephropathy, fatty liver, and dyslipidemia, which are representative complications, is associated with an increase in TNF-α protein positive cells in CD34/CD106 bone marrow progenitor cells in peripheral blood. On the other hand, in the type 2 diabetic patients, onset of diabetic neuropathy, retinopathy, nephropathy, fatty liver, and dyslipidemia is associated with an increase in TNF-α protein positive cells and an increase in proinsulin positive cells in CD3/CD106 bone marrow progenitor cells in peripheral blood.

(Discussion and Expectation of the Conclusion)

1) Non-diabetes mellitus has CD34/CD106 bone marrow progenitor cells which express insulin mRNA and TNF-α mRNA, although only slightly, in blood. It is expected that these cells function as an endothelial cell which presents an autoantigen when homing to the pancreatic islet.

2) In type 2 diabetes mellitus, these cells (CD34/CD106 bone marrow progenitor cells expressing insulin mRNA and TNF-α mRNA) are present in the bone marrow and blood due to hyperglycemia. It is expected that these cells cause insulin resistance or various complications by prior expression of proinsulin and TNF-α protein and, concurrently, differentiation into a vascular endothelium with an abnormal function or possession of an abnormal cell fusion ability.

Research Plan 2: Therapy of Diabetes Mellitus Targeting a Bone Marrow-Derived Abnormal Hematopoietic Stem Cell

(Subject)

in accordance with Research Plan 1, 280 diabetic patients (140 patients with type 1 diabetes mellitus and 140 patients with type 2 diabetes mellitus) are registered in Shiga University of Medical Science and the collaborative research facility. It is believed that both type 1 diabetes mellitus and type 2 diabetes mellitus do not heal upon disease onset. However, it is known that the honeymoon phase, in which temporary remission is exhibited by strict blood glucose control using insulin, appears in type 1 diabetes mellitus. Although the period of the phase varies depending on the report, it has been reported that the period is generally 1 month to 13 years (Wallensteen M, Dahiquist G, Persson B, Landin-Olsson: M, Lernmark A, Sundkvist G, Thalme B (1988) Factors influencing the magnitude, duration, and rate off all of β-cell function in type 1 (insulin-dependent) diabetic children followed for two years from their clinical diagnosis. Diabetologia 31: 664-669). Thus, it may be difficult to discern whether carrying out the present therapy plan has resulted in the honeymoon phase or healing of the disease itself. For type 2 diabetes mellitus, it has been reported that insulin resistance becomes mild by strict control (H. E. Lebovitz (2001) Insulin resistance: definition and consequences. Clin Endocrinol Diabetes 109 Suppl 2: S135-S148). This may result in improvement in the amount of a therapeutic drug such as insulin or oral agents as well as the endogenous insulin secretion ability. Thus, in the present therapy research, it is necessary to prepare a group to be treated by insulin alone and a group to be treated by a novel therapeutic method to compare and study these two groups for both type 1 and type 2.

(Research Method)

Medical questions are asked to the subjects, the height and body weight of the subjects are measured, a blood test and a urine test are performed, and the presence or absence of diabetes mellitus is determined. Mononuclear cells are extracted from the collected blood, CD34-labelled bone marrow progenitor cells are collected and fixed, and the form and expressed protein are identified by immunostaining. Further, after mRNA is extracted, cDNA is prepared and the amount of expression of mRNA is quantified by quantitative PCR. Specifically, the amount of expression of TNF-α mRNA and insulin mRNA or the like is measured in CD34 positive and CD106 positive bone marrow progenitor cells in peripheral blood. The presence or absence of expression of protein in these cells is also measured. Association between the presence or absence of a diabetic complication in the diabetic patients and blood glucose control is also analyzed. A nerve conduction velocity test, an electrocardiogram R-R interval test, an ophthalmoscopy, a urinary albumin excretion rate, quantification of the amount of intraperitoneal fat using abdominal CT, a blood lipid test, a carotid artery echo test, and a lower limb artery echo test are performed to check the presence or absence of diabetic neuropathy, retinopathy, nephropathy, fatty liver, and dyslipidemia, which are representative complications, and macrovasculopathy.

The type 1 cases are classified at random into seven groups each having 20 cases. The type 2 cases are classified at random into seven groups each having 20 cases. 20 cases of each of type 1 and type 2 are controlled for three months with insulin therapy alone (control group). For 60 cases among the remaining 120 cases, each of the following three types of therapies is started for 20 cases simultaneously with the start of insulin therapy (therapy group without the migration agent). 1) An anti-TNF-α antibody (40 mg/kg of adalimumab or 3 mg/kg of infliximab), 2) an anti-CD106 antibody (0.8 mg/kg), or 3) trichostatin (0.5 mg/kg) is intravenously administered once a week, and the therapy is continued for twelve weeks. For the remaining 60 cases, each of the following three types of therapies is started for 20 cases simultaneously with the start of insulin therapy (therapy group with the migration agent). 1) An anti-TNF-α antibody (40 mg/kg of adalimumab or 3 mg/kg of infliximab), 2) an anti-CD106 antibody (0.8 mg/kg), or 3) trichostatin (0.5 mg/kg) is intravenously administered together with the cell migration agents, Groβ (100 μg/kg) Plerixafor (0.24 mg/kg), once a week, and the therapy is continued for twelve weeks.

(Method for Determining the Therapeutic Effect)

The patients perform self-monitoring of blood glucose 6 times a day. The target of blood glucose control is to achieve a pre-meal blood glucose level which is 140 mg/dl or less and a blood glucose level after 2 hours post-meal which is 200 mg/dl or less by insulin therapy. The amount of insulin is actively increased or decreased so as to satisfy the control criteria. Ultimately, the therapy research period ends with a dosage of insulin required for blood glucose control in week 12.

The therapeutic effect is determined by follow-up determination prior to the therapy, at the time of the end of the therapy, and after 3 months, 6 months, 9 months, and months from the end of the therapy, considering the following determination items as the therapeutic effect.

(Determination Items)

(A) Effect of Eliminating an Abnormal Hematopoietic Stem Cell

Expressed protein (such as CD34, proinsulin, TNF-α, or CD106) and expressed gene (such as CD34 mRNA, insulin mRNA, TNF-α mRNA, or CD106 mRNA) in CD34/CD10 bone marrow progenitor cells in peripheral blood are quantified.

(B) Effect of Healing Diabetes Mellitus

Blood glucose level, HbA1c, urinary CPR, lipid, and insulin secretion ability with a glucagon loading test are measured prior to and after the therapy. A pancreatic islet-associated antibody is measured, and it is revealed whether the antibody is eliminated.

(C) Therapeutic Effect on a Complication

The presence or absence and change of diabetic neuropathy, retinopathy, nephropathy, fatty liver, dyslipidemia, and macrovasculopathy, which are representative complications, are compared and studied prior to and after the therapy.

(Prediction of Results)

1) Therapy with insulin alone reveals the following.

(A) Abnormal hematopoietic stem cells are not eliminated in both type 1 diabetes mellitus and type 2 diabetes mellitus.

(B) The effect of healing diabetes mellitus is not observed.

(C) The therapeutic effect on a complication is observed to some extent, but the complication does not heal.

2) Regardless of the presence or absence of a cell migration agent, novel therapy reveals the following.

(A) Abnormal hematopoietic stem cells are eliminated in both type 1 diabetes mellitus and type 2 diabetes mellitus.

(B) Both type 1 diabetes mellitus and type 2 diabetes mellitus heal once.

(C) Progression of a complication is stopped and an obvious therapeutic effect is observed in both type 1 diabetes mellitus and type 2 diabetes mellitus.

3) Novel therapy with the addition of cell migration agents reveals the following.

(A) Abnormal hematopoietic stem cells are eliminated earlier in both type 1 diabetes mellitus and type 2 diabetes mellitus by the novel therapy with a migration agent added thereto compared to the case without the migration agent.

(B) The cure rate of both type 1 diabetes mellitus and type 2 diabetes mellitus is improved by the novel therapy with a migration agent added thereto compared to the case without the migration agent.

(C) The cure rate of complications is improved in both type 1 diabetes mellitus and type 2 diabetes mellitus by the novel therapy with a migration agent added thereto compared to the case without the migration agent.

(Discussion and Expectation of the Conclusion)

1) For type 1, it is possible that the blood glucose control effect results in the honeymoon phase even when diabetes mellitus is treated with insulin alone. However, in this case, the remission will eventually end, and there will be deficiency in the insulin secretion ability again.

2) For type 2 diabetes mellitus, it is possible that the blood glucose control is improved by therapy with insulin alone and therapy with insulin is no longer necessary. However, since it is not possible to eliminate the abnormal hematopoietic stem cells, diabetes mellitus does not heal.

3) Even when type 1 diabetes mellitus heals by the novel therapy with the addition of cell migration agents, there remains a possibility that autoimmunity, which was a cause of production of an autoantibody, may recur. However, the novel therapy with the addition of cell migration agents may be performed again.

4) Even when type 2 diabetes mellitus completely heals by the novel therapy with the addition of cell migration agents, abnormal hematopoietic stem cells may appear if hyperglycemia is recurred again due to an excessive intake of energy or a lack of exercise. Diabetes mellitus also can be treated in this case if the novel therapy with the addition of cell migration agents is performed again.

(Note)

As described above, the present disclosure is exemplified by the use of its preferred embodiments. However, it is understood that the scope of the present invention should be interpreted solely based on the Claims. It is also understood that any patent, any patent application, and any references cited herein should be incorporated herein by reference in the same manner as the contents are specifically described herein.

The present application claims priority to Japanese Patent Application No. 2019-191369 filed to the Japan Patent Office on Oct. 18, 2019. The entire content thereof is incorporated herein by reference.

### INDUSTRIAL APPLICABILITY

The present disclosure provides improvements in the therapy for diabetes mellitus targeting stem cells, and based on this finding, provides new approaches for therapy and prevention of diabetes mellitus and/or a disease, disorder, and/or symptom associated with diabetes mellitus and diagnosis of diabetes mellitus and/or a disease, disorder, and/or symptom associated with diabetes mellitus or a risk thereof.

1.-16. (canceled)

17. A method for treating and/or preventing diabetes mellitus or a disease, disorder, and/or symptom associated with diabetes mellitus in a subject, the method comprising: administering an effective amount of an agent that reduces or eliminates an abnormal hematopoietic stem cell (HSC) and a stem cell migration agent to the subject.

18. A method for using a migration and/or residual state of an abnormal hematopoietic stem cell (HSC) as an indicator of treatment for treating and/or preventing diabetes mellitus or diabetes mellitus and/or a disease, disorder, and/or symptom associated with diabetes mellitus in a subject, the method comprising: detecting the migration and/or residual state of the abnormal hematopoietic stem cell (HSC) in the subject.

19. The method of claim 17, wherein the abnormal HSC is a cell in which a gene or protein selected from the group consisting of CD106 and a functional equivalent thereof is not expressed and/or does not function at a normal level.

20. The method of claim 19, wherein the expression which is not at a normal level is overexpression.

21. The method of claim 17, wherein the suppressing agent comprises at least one selected from the group consisting of an anti-CD106 antibody or a functional variant thereof.

22. The method of claim 19, wherein the abnormal HSC is a cell in which a gene or protein selected from the group consisting of tumor necrosis factor alpha (TNF-α), histone deacetylase (HDAC), and proinsulin is further not expressed at a normal level.

23. The method of claim 17, wherein the suppressing agent comprises at least one selected from the group consisting of an anti-TNF-α antibody or a functional variant thereof and an HDAC inhibiting agent.

24. The method of claim 17, wherein the disease, disorder, and/or symptom comprises a diabetic complication.

25. The method of claim 17, wherein the disease, disorder, and/or symptom is selected from the group consisting of neuropathy, nephropathy, hepatopathy, retinopathy, fatty liver, gastrointestinal disorder, delayed bone fracture healing, eating disorder, and dermatopathy.

26. The method of claim 17, wherein the stem cell migration agent has an ability to cause the abnormal HSC to migrate from a niche.

27. The method of claim 17, wherein the stem cell migration agent comprises at least one agent selected from the group consisting of a CXCR4 antagonizing agent, a CXCR2 stimulating agent, an epidermal growth factor receptor (EGFR) inhibiting agent, and a granulocyte colony stimulating factor (G-CSF) agent.

28. The method of claim 17, wherein the stem cell migration agent comprises at least one selected from the group consisting of Plerixafor, GROβ2 (MIP2), Gefitinib, Erlotinib, Afatinib, Osimertinib, Filgrastim, Nartograstim, Lenograstim, and Pegfilgrastim.

29. The method of claim 18, wherein the migration and/or residual state is a migration from a niche of a bone marrow and/or a residual state at the niche of the bone marrow.

30. The method of claim 18, wherein the migration detection agent comprises a detection agent for CD106 or a functional equivalent thereof.
